(19) United States
(12) Patent Application Publication
(10) Pub. No.: US 2023/0012931 A1
(43) Pub. Date: Jan. 19, 2023

(54) MOBILE DEVICE HAVING CLIP ANTENNA

(71) Applicant: ACS DIAGNOSTICS, INC., Irvine, CA (US)

(72) Inventors: Loren A. Manera, Irvine, CA (US); Francis W. Walker, JR., Laguna Niguel, CA (US)

(73) Assignee: ACS DIAGNOSTICS, INC., Irvine, CA (US)

(21) Appl. No.: 17/813,266

(22) Filed: Jul. 18, 2022

Related U.S. Application Data
(60) Provisional application No. 63/222,624, filed on Jul. 16, 2021.

(51) Int. Cl. A61B 5/332; H01Q 1/52; A61B 5/00

(52) U.S. Cl. CPC A61B 5/332; H01Q 1/526; A61B 5/0022; A61B 5/6802; A61B 2560/0468

(57) ABSTRACT

An electrocardiogram recording device can include a housing, a clip, a modem, and an antenna. The housing can contain RF shielding, which can be arranged to minimize RF signal transmissions in one direction while not limiting RF signal transmission in an opposite direction. The clip can be attached to the housing. An antenna can be integrated within the clip. The modem can be a cellular data modem.

### TECHNICAL FIELD

The present disclosure relates generally to the field of portable health monitors, and more specifically to devices with a clip having an antenna. Embodiments can have specific applicability to battery-operated multi-function monitors and/or transmitters, but can also be more widely applicable to other types of portable communication devices.

### BACKGROUND

Handheld mobile telemetry medical devices can be capable of communicating directly and/or indirectly with cell towers. Largely, body-worn systems have relied on indirect communication with cell infrastructure. For example, they have utilized intermediary gateway devices (such as a smart phone) to communicate via low-power RF signals (such as Bluetooth) and to communicate via relatively higher-power RF signals with cell-phone infrastructure. Direct communication between the medical devices and the cell infrastructure can achieved. However, problems can arise when such devices are used in close proximity to a person. Accordingly, embodiments herein seek to address and overcome such problems.

### SUMMARY

An aspect of the invention can include an electrocardiogram recording device. The electrocardiogram recording device can include a housing, a clip, an antenna integrated within the clip, and/or a modem. The clip attached to the housing. The antenna can be integrated within the clip.

In an embodiment, a housing is a clam-shell housing.

In another embodiment, shielding can be disposed on an internal surface of a housing. The housing can be plastic. The shielding can be conductive shielding applied to the housing.

In yet another embodiment, a modem is a cellular data modem. The antenna can be ungrounded.

In an embodiment, a housing can include one or more ports. The ports can be configured to connect to an ECG patch.

### BRIEF DESCRIPTION OF THE DRAWINGS

The present invention is further articulated in the detailed description which follows, in reference to the noted plurality of drawings by way of non-limiting examples of certain embodiments of the present invention, in which like numerals represent like elements throughout the several views of the drawings, and wherein:

FIG. 1 illustrates an ECG recording device having an antenna disposed in a clip.

FIG. 2 illustrates a view of an internal chipset for an ECG recording device.

FIG. 3 illustrates another view of an internal chipset for an ECG recording device.

### DETAILED DESCRIPTION

A detailed explanation of the system, method, and exemplary embodiments of the present invention are articulated below. Exemplary embodiments described, shown, and/or disclosed herein are not intended to limit the invention, but rather, are intended to instruct one of ordinary skill in the art as to various aspects of the invention. Other embodiments can be practiced and/or implemented without departing from the scope and spirit of the invention.

Mobile cardiac outpatient telemetry (MCOT or MCT) can provides continuous outpatient electrocardiographic monitoring in real time. Handheld mobile telemetry medical devices (of which MCOT and event recorders are examples) that are capable of communicating directly to cell towers have been enabled by recent technological developments in low-power cell data modem communications. Formerly, body-worn systems more typically used a gateway device, such as a standard cellular smart-phone, to receive the Bluetooth (or equivalent very low-power RF signal) and then re-transmit it to the cell-phone infrastructure.

At the same time, electrocardiogram (ECG) recording advances have pushed the marketplace toward the requirement for very physically small recorders, such as “patch” devices. An essential problem with patch devices is that their electronics are quite proximate to the patient's body (typically right above the sternum, with electronics only a few millimeters away from the body.) A patch recorder device housing a cell-phone data modem can subject the wearer to unacceptable levels of RF energy, requiring strenuous RF mitigation strategies to be employed in order to satisfy acceptable specific absorption rate (SAR) standards.

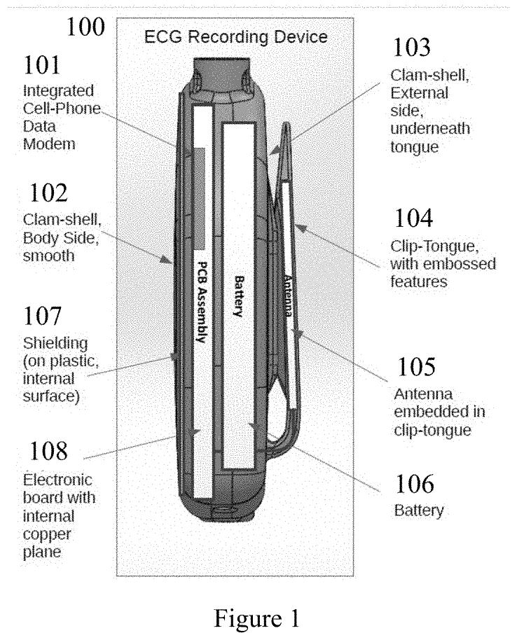

Embodiments herein present a technological solution to the SAR issue posed by direct-to-cell-tower communications from a close-to-body recorder device is to house the recorder such that while it may rest on the body, its antenna (which radiates the RF signal) is at a physically controlled distance such that it is in an offset position from the body. One such solution is depicted in FIG. 1.

FIG. 1 illustrates an ECG recording device (100). The recording device can include a housing. As depicted, the housing can be a clam-shell housing (102, 103). The housing can be mass-produced from, for example, a plastic such as ABS plastic or polycarbonate, and may have metal components. The recording device can include an integrated cell-phone data modem (101) and a clip-tongue (104). The antenna can be placed in a pre-molded and/or machined cavity within the tongue area, e.g. to control its position. Various cell-phone data modems can be utilized, such as a u-blox SARA-410M and/or Telit ME910.

The recording device can include an antenna (105). As depicted, the antenna can be embedded in the clip-tongue (104) of the housing. The recording device can include RF shielding (107). The RF shielding can be arranged to minimize RF signal transmissions in one direction while not limiting RF signal transmission in an opposite direction. As depicted in FIG. 1, the shielding can be disposed on an internal surface of the housing, opposite the antenna (105). The shielding can be conductive shielding that is applied to the clam-shell plastic. The shielding can be made of various conductors, including for example copper, aluminum, nickel, silver, and/or tin-plated steel. The shielding can be applied and secured to the plastic by adhesive, such as by utilizing, e.g., RF shielding tape, but other types of shielding are contemplated, such as foil and/or plates.

Internal to the housing, the recording device can include a battery (106) and a circuit board (108). A circuit board can be a printed circuit board (PCB) and/or can include a conductive plane, such as a copper or a low magnetic reluctance plate. The design and configuration of the circuit board and associated shielding can affect the operation of the antenna, and thereby the operation can be optimized.

A safety offset distance for the antenna (105) can be provided by the stack-up width dimension of the clam-shell housing (102 & 103) and by the clip-tongue (104). The cell modem can be integrated (for example by soldering) with the circuit board assembly. It should be noted that the battery and/or other componentry can provide some or all of the radiation shielding. The specific placement of and dimensions of the battery and the shielding can be based on the safety design requirements.

The antenna can be embedded within the clip-tongue and/or placed such that the near-field radiation pattern in the direction of the wearer's body can be absorbed and/or reflected by the recorder's battery and is additionally reflected by the copper and other metal planes within the electronic circuit board and the shielding placed upon the plastic clam-shell package, the latter being closest to the wearer's skin.

Medical providers can issue such recorders and can instruct patients to properly place the smooth portion of clam-shell housing (102) of the device near their skin, with the clip-tongue (104) away from the body. The shape of the device and/or optional embossing on the clip-tongue and/or clam-shell housing (103) can make wearing the device in a backward orientation uncomfortable, thus providing encouragement for the patient to intuitively understand how to place the device properly.

Commercial off-the-shelf (COTS) antennas are widely available with various radiation patterns and physical sizes such that the shielding effect can be achieved. Integral to an approach can be the use of COTS modified by utilizing ungrounded cell antennas. This type of antenna does not require the printed circuit board ground plane to be in close proximity to the radiating element, i.e., the portion that is placed on the clip-tongue in this design. As an example, the Siretta Echo 1A provides an antenna that is both ungrounded and physically relatively small in comparison with the clip housing and underlying shielding, thereby optimizing offset and shielding effectiveness.

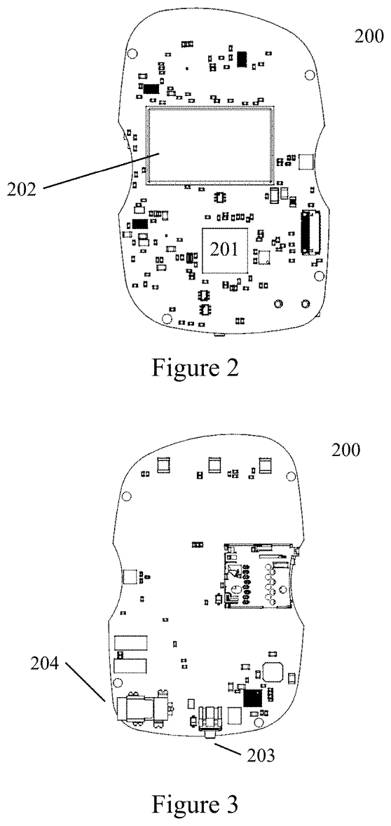

FIGS. 2 and 3 illustrate a circuit board assembly for a specific embodiment. As shown in FIG. 1, the assembly can be implemented on a printed circuit board, or PCB (200). By way of illustrative and nonlimiting example of a preferred embodiment, the custom PCB assembly (200) can have x-y dimensions of approximately 66 mm×43 mm, where the antenna x-y size is approximately 35 mm×6 mm and can be distanced greater than 10 mm from a user's body. The PCB can include a processor (201) and a cellular data modem (202). FIG. 2 illustrates the reverse side of the same PCB assembly (200). The board can include various physical ports. As shown, for example, the board can include a power jack (203) and connector (204) for leads, such as to a patch and/or conventional leads.

By selecting appropriate dimensions for the antenna, clip-tongue, battery thickness, and clam-shell shielding, suitable mitigation of RF energy to levels well below the required SAR standards can be achieved.

The clip antenna device described herein can be applied to a patch device. For example, such embodiments can be implemented by embedding the antenna in a clip structure (similar to a tie-clip) but configured such that there is an exterior wired connection to from the patch to the antenna. This approach is practicable using the shielding concept described herein but is not as physically robust as the housing of FIG. 1.

All of the systems disclosed herein can be made and implemented without undue experimentation in light of the present disclosure. While the apparatus of this invention have been described in terms of preferred embodiments, it will be apparent to those of skill in the art that variations may be applied to the systems and apparatus described herein without departing from the concept, spirit and scope or the invention. In addition, from the foregoing it will be seen that this invention is one well adapted to attain all the ends and objects set forth above, together with other advantages. It will be understood that certain features and sub-combinations are of utility and may be employed without reference to other features and sub-combinations. This is contemplated and within the scope of the appended claims. All such similar substitutes and modifications apparent to those skilled in the art are deemed to be within the spirit and scope of the invention as defined by the appended claims.

1. An electrocardiogram recording device, comprising:
a housing;
a clip attached to the housing;
a modem;
an antenna integrated within the clip.

2. The electrocardiogram recording device of claim 1, wherein the housing is a clam-shell housing.

3. The electrocardiogram recording device of claim 1, further comprising shielding disposed on an internal surface of the housing.

4. The electrocardiogram recording device of claim 3, wherein the housing is plastic, and wherein the shielding is conductive shielding applied to the housing.

5. The electrocardiogram recording device of claim 1, wherein the modem is a cellular data modem.

6. The electrocardiogram recording device of claim 5, wherein the antenna is ungrounded.

7. The electrocardiogram recording device of claim 1, wherein the housing includes one or more ports configured to connect to an ECG patch.
